SEQUENCE LISTING (11 sequences)

SEQ ID NO: 1 - DNA, 351 bp, Artificial Sequence
  all or portions of recombinant immunotoxin or its encoding sequence
gaggtgcagctggtggagtctgggggaggcttagtgcagcctggaaggtccctgaaactctcctgtgcagcctcaggattcactttcagtaactatggcatggcctgggtccgccagactccaacgaagtgcctggagtggatcgcatccattagtgctggtggtgataaaacttactatggagactccgtgaagggccgattcagtatctccagagataatgcaaaaaccacccactacttgcaaatggacagtctgaggtctgaggacacggccacttattactgtgcaaaaacttcccgggtatattttgattactggggccaaggagtcatggtcacagtctcctca

SEQ ID NO: 2 - DNA, 342 bp, Artificial Sequence
  all or portions of recombinant immunotoxin or its encoding sequence
gcccagtttgtgcttactcagccaaactctgtgtctacgaatctcggaagcacagtcaaactgtcttgtaagcgcagcactggtaacattggaagcaattatgtgaactggtaccagcagcatgagggaagatctcccaccactatgatttatagggatgataagagaccagatggagttcctgacaggttctctggctccattgacagatcttccaactcagccctcctgacaatcaataatgtgcagactgaagatgaagctgactacttctgtcactcttacagtagtggtattgttttcggttgcggaaccaagctcactgtcctaggtcagcccaag

SEQ ID NO: 3 - protein, 117 aa, Artificial Sequence
  all or portions of recombinant immunotoxin or its encoding sequence
EVQLVESGGGLVQPGRSLKLSCAASGFTFSNYGMAWVRQTPTKCLEWIASISAGGDKTYYGDSVKGRFSISRDNAKTTHYLQMDSLRSEDTATYYCAKTSRVYFDYWGQGVMVTVSS

SEQ ID NO: 4 - protein, 114 aa, Artificial Sequence
  all or portions of recombinant immunotoxin or its encoding sequence
AQFVLTQPNSVSTNLGSTVKLSCKRSTGNIGSNYVNWYQQHEGRSPTTMIYRDDKRPDGVPDRFSGSIDRSSNSALLTINNVQTEDEADYFCHSYSSGIVFGCGTKLTVLGQPK

SEQ ID NO: 5 - DNA, 45 bp, Artificial Sequence
  all or portions of recombinant immunotoxin or its encoding sequence
ggcggagggggatccggtggtggcggatctggaggtggcggcagc

SEQ ID NO: 6 - protein, 5 aa, Artificial Sequence
  all or portions of recombinant immunotoxin or its encoding sequence
NYGMA

SEQ ID NO: 7 - protein, 17 aa, Artificial Sequence
  all or portions of recombinant immunotoxin or its encoding sequence
SISAGGDKTYYGDSVKG

SEQ ID NO: 8 - protein, 9 aa, Artificial Sequence
  all or portions of recombinant immunotoxin or its encoding sequence
KTSRVYFDY

SEQ ID NO: 9 - protein, 13 aa, Artificial Sequence
  all or portions of recombinant immunotoxin or its encoding sequence
KRSTGNIGSNYVN

SEQ ID NO: 10 - protein, 7 aa, Artificial Sequence
  all or portions of recombinant immunotoxin or its encoding sequence
RDDKRPD

SEQ ID NO: 11 - protein, 8 aa, Artificial Sequence
  all or portions of recombinant immunotoxin or its encoding sequence
HSYSSGIV

(19) United States
(12) Patent Application Publication
(10) Pub. No.: US 2014/0017266 A1
(43) Pub. Date: Jan. 16, 2014

(54) ANTI-PODOPLANIN ANTIBODIES AND METHODS OF USE

(71) Applicants: Darell Bigner, Mebane, NC (US); Chien-Tsun Kuan, Cary, NC (US); Ira H. Pastan, Potomac, MD (US); Vidyalakshmi Chandramohan, Durham, NC (US)

(72) Inventors: Darell Bigner, Mebane, NC (US); Chien-Tsun Kuan, Cary, NC (US); Ira H. Pastan, Potomac, MD (US); Vidyalakshmi Chandramohan, Durham, NC (US)

(73) Assignees: THE GOVERNMENT OF THE UNITED STATES, AS REPRESENTED BY THE SECRETARY OF HHS, NIH, ROCKVILLE, MD (US); DUKE UNIVERSITY, DURHAM, NC (US)

(21) Appl. No.: 13/991,308

(22) PCT Filed: Dec. 5, 2011
(86) PCT No.: PCT/US11/63284

§ 371 Date: Sep. 17, 2013

Related U.S. Application Data
(60) Provisional application No. 61/419,327, filed on Dec. 3, 2010.

(51) Int. Cl. C07K 16/18; C07K 14/21

(52) U.S. Cl. CPC C07K 16/18; C07K 14/21
USPC 424/183.1; 530/391.7; 536/23.53; 435/69.6; 435/375

(57) ABSTRACT

Recombinant scFv-immunotoxins target tumor cells expressing human podoplanin but not podoplanin-negative or normal cells. The immunotoxins can be used for treatment of malignant glioma patients or any malignant tumor expressing podoplanin. One such immunotoxin comprises a modified Pseudomonas exotoxin (PE38) attached to the scFv antibody fragment. This immunotoxin can be used as a therapeutic drug for the treatment of malignant gliomas and other cancers.

The invention was made using funds from the U.S. Government. Certain rights are retained in this invention under the terms of grant no. CA11898 from the National Institutes of Health.

### BACKGROUND OF THE INVENTION

Podoplanin/Aggrus is a mucin-like sialoglycoprotein that is highly expressed in malignant gliomas. Podoplanin has been reported to be a marker to enrich tumor-initiating cells, which are thought to resist conventional therapies and to be responsible for relapse. There is a continuing need in the art for agents which can be used to successfully treat malignant tumors such as gliomas, especially those that resist conventional therapies.

### BRIEF DESCRIPTION OF THE INVENTION

One aspect of the present invention is an immunotoxin which consists of a single polypeptide that binds to podoplanin and which is cytotoxic to cells expressing podoplanin. The immunotoxin comprises an antibody heavy chain variable (“V_{H}”) region and an antibody light chain variable (“V_{L}”) region. Each region comprises three complementarity determining regions (“CDRs”). The CDRs of each region are numbered sequentially CDR1 to CDR3 starting from the amino terminus CDR1, CDR2, and CDR3 of the V_{H} are shown in SEQ ID NO: 6, 7, and 8 respectively. CDR1, CDR2, and CDR3 of the V_{L} are shown in SEQ ID NO: 9, 10 and 11. The immunotoxin further comprises a Pseudomonas exotoxin or cytotoxic fragment thereof (“PE”).

Another aspect of the invention is a deoxyribonucleic acid molecule which encodes an immunotoxin. The immunotoxin consists of a single polypeptide that binds to podoplanin and which is cytotoxic to cells expressing podoplanin. The immunotoxin comprises an antibody heavy chain variable (“V_{H}”) region and an antibody light chain variable (“V_{L}”) region. Each region comprises three complementarity determining regions (“CDRs”). The CDRs of each region are numbered sequentially CDR1 to CDR3 starting from the amino terminus CDR1, CDR2, and CDR3 of the V_{H} are shown in SEQ ID NO: 6, 7, and 8 respectively. CDR1, CDR2, and CDR3 of the V_{L} are shown in SEQ ID NO: 9, 10 and 11. The immunotoxin further comprises a Pseudomonas exotoxin or cytotoxic fragment thereof (“PE”).

Another aspect of the invention is a method of making an immunotoxin. A cell which comprises a deoxyribonucleic acid molecule is cultured in a cell culture medium, and the immunotoxin is collected from the cultured cells or cell culture medium. The deoxyribonucleic acid molecule encodes an immunotoxin. The immunotoxin consists of a single polypeptide that binds to podoplanin and which is cytotoxic to cells expressing podoplanin. The immunotoxin comprises an antibody heavy chain variable (“V_{H}”) region and an antibody light chain variable (“V_{L}”) region. Each region comprises three complementarity determining regions (“CDRs”). The CDRs of each region are numbered sequentially CDR1 to CDR3 starting from the amino terminus CDR1, CDR2, and CDR3 of the V_{H} are shown in SEQ ID NO: 6, 7, and 8 respectively. CDR1, CDR2, and CDR3 of the V_{L} are shown in SEQ ID NO: 9, 10 and 11. The immunotoxin further comprises a Pseudomonas exotoxin or cytotoxic fragment thereof (“PE”).

Another aspect of the invention is a method for inhibiting the growth of malignant cells expressing podoplanin on their cell surface. The malignant cells are contacted with an immunotoxin, whereby the immunotoxin inhibits the growth of the cells. The immunotoxin comprises an antibody heavy chain variable (“V_{H}”) region and an antibody light chain variable (“V_{L}”) region. Each region comprises three complementarity determining regions (“CDRs”). The CDRs of each region are numbered sequentially CDR1 to CDR3 starting from the amino terminus. CDR1, CDR2, and CDR3 of the V_{H} are shown in SEQ ID NO: 6, 7, and 8 respectively. CDR1, CDR2, and CDR3 of the V_{L} are shown in SEQ ID NO: 9, 10 and 11. The immunotoxin further comprises a Pseudomonas exotoxin or cytotoxic fragment thereof (“PE”).

These and other embodiments and aspects provide the art with therapeutic tools for treating tumors expressing podoplanin.

### BRIEF DESCRIPTION OF THE DRAWINGS

FIG. 1A-1L shows flow cytometric analysis of brain tumor xenografts to determine reactivity of the NZ-1 mAb.

FIG. 2 shows the DNA sequence of dsNZ-1 scFv. (SEQ ID NO: 1, 5, and 2.)

FIG. 3A-3B shows the amino acid sequence of dsNZ-1 scFv. (SEQ ID NO: 3 and 4, which include SEQ ID NOS: 6-8 and 9-11, respectively.)

FIG. 4 is a schematic showing the structure of dsNZ-1-PE38 KDEL immunotoxin.

FIG. 5A-5F are graphs showing protein synthesis inhibition of brain tumor xenografts by dsNZ-1-PE38 KDEL immunotoxin.

FIG. 6 shows tumor volume growth inhibition in a medulloblastoma xengograft (D283MED) by an immunotoxin in disulfide stabilized and not stabilized form.

FIG. 7 shows tumor volume growth inhibition in a glioblastoma multiforme xenograft (D2159MG) by an immunotoxin in disulfide stabilized and not stabilized form.

### DETAILED DESCRIPTION OF THE INVENTION

An immunotoxin according to the invention consists of a single polypeptide that binds to podoplanin and is cytotoxic to cells expressing podoplanin. It comprises an antibody heavy chain variable (“V_{H}”) region and an antibody light chain variable (“V_{L}”) region. Each region comprises three complementarity determining regions (“CDRs”). The CDRs of each region are numbered sequentially CDR1 to CDR3 starting from the amino terminus CDR1, CDR2, and CDR3 of the V_{H} are shown in SEQ ID NO: 6, 7, and 8 respectively, and CDR1, CDR2, and CDR3 of the V_{L} are as shown in SEQ ID NO: 9, 10 and 11. The immunotoxin further comprises a Pseudomonas exotoxin or cytotoxic fragment thereof (“PE”).

The immunotoxin may optionally have a V_{H} chain as shown in SEQ ID NO: 3. The immunotoxin may optionally have a V_{L} chain as shown in SEQ ID NO: 4. The immunotoxin may comprises an exotoxin which is PE38, optionally the PE38 KDEL form of the exotoxin. The immunotoxin may optionally be disulfide stabilized. Such disulfide stabilization may optionally be a disulfide bridge between a cysteine residue in the V_{H} at residue 44 of SEQ ID NO: 3 and a cysteine residue in the V_{L} at residue 103 of SEQ ID NO: 4. The immunotoxin may optionally comprise a linker segment between the V_{H} and the V_{L}. In one particular example, the immunotoxin is encoded by a nucleic acid molecule having a sequence in the 5′ to 3′ direction of SEQ ID NO: 1, 5, and 2.

A deoxyribonucleic acid molecule encoding the immunotoxin may be used, for example to make the immuntoxin. The manufacturing may be done in cells in culture, for example, or cells in a host animal, or in an ultimate recipient to be treated. The deoxyribonucleic acid molecule may optionally comprises a sequence in the 5′ to 3′ direction of SEQ ID NO: 1, SEQ ID NO: 5, and SEQ ID NO: 2. Other coding sequences of the same immunotoxin may be used. Other coding sequences of an alternate immunotoxin having the same CDR regions may be used.

One method of making an immunotoxin involves, culturing a cell which comprises a deoxyribonucleic acid molecule in a cell culture medium, and collecting the immunotoxin from the cultured cells or cell culture medium. The deoxyribonucleic acid molecule encoding the immunotoxin may be used, for example to make the immuntoxin. The manufacturing may be done in cells in culture, for example, or cells in a host animal, or in an ultimate recipient to be treated. The deoxyribonucleic acid molecule may optionally comprises a sequence in the 5′ to 3′ direction of SEQ ID NO: 1, SEQ ID NO: 5, and SEQ ID NO: 2. Other coding sequences of the same immunotoxin may be used. Other coding sequences of an alternate immunotoxin having the same CDR regions may be used.

The immunotoxins can be used to inhibit or treat cells in culture or in vivo. They typically will effect preferentially growth of malignant cells expressing podoplanin on their cell surface relative to cells which do not express podoplanin on their cell surfaces. The malignant cells are contacted with an immunotoxin. The immunotoxin inhibits the growth of the cells. The cells which may be contacted with the mmunotoxin include without limitation astrocytoma cells, glioma cells, glioblastoma multiforme cells, melanoma cells and ependymoma cells. Any of the forms and variants of the immunotoxin described may be used for the contacting and/or treating.

### DEFINITIONS

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the disclosure which can be had by reference to the specification as a whole. Accordingly, the terms defined immediately below are more fully defined by reference to the specification in its entirety.

The term “podoplanin” or PDPN includes reference to the transmembrane sialoglycoprotein present on lymphatic endothelial cells, and whose expression has been shown to be upregulated in several cancers, including glioblastoma multiforme (see, e.g., Rica, M. et al. (2008) Anticancer Res. 28(5B):2997-3006; Mishima, K. et al. (2006) Acta Neuropathol. 111(5):483-488, both of which are incorporated by reference). Podoplanin also refers to podoplanin proteins or peptides which remain intracellular as well as secreted and/or isolated extracellular protein.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies), heteroconjugate antibodies (e.g., bispecific antibodies) and recombinant single chain Fv fragments (scFv), disulfide stabilized (dsFv) Fv fragments (See, U.S. Ser. No. 08/077,252, incorporated herein by reference), or pFv fragments (See, U.S. Provisional Patent Applications 60/042,350 and 60/048,848, both of which are incorporated herein by reference.). The term “antibody” also includes antigen binding forms of antibodies (e.g., Fab′, F(ab′)₂, Fab, Fv and rIgG (See also, Pierce Catalog and Handbook, 1994-1995 (Pierce Chemical Co., Rockford, Ill.).

An antibody immunologically reactive with a particular antigen can be generated by recombinant methods such as selection of libraries of recombinant antibodies in phage or similar vectors (See, e.g., Huse, et al., Science 246:1275-1281 (1989); Ward, et al., Nature 341:544-546 (1989); and Vaughan, et al., Nature Biotech. 14:309-314 (1996)).

Typically, an immunoglobulin has a heavy and light chain. Each heavy and light chain contains a constant region and a variable region. Light and heavy chain variable regions contain a “framework” region interrupted by three hypervariable regions, also called complementarity-determining regions or CDRs. The extent of the framework region and CDRs have been defined (see, SEQUENCES OF PROTEINS OF IMMUNOLOGICAL INTEREST, Kabat, E., et al., U.S. Department of Health and Human Services, (1987); which is incorporated herein by reference). The sequences of the framework regions of different light or heavy chains are relatively conserved within a species. The framework region of an antibody, that is the combined framework regions of the constituent light and heavy chains, serves to position and align the CDRs in three dimensional space. The CDRs are primarily responsible for binding to an epitope of an antigen. The CDRs are typically referred to as CDR1, CDR2, and CDR3, numbered sequentially starting from the N-terminus.

The phrase “single chain Fv” or “scFv” refers to an antibody in which the heavy chain and the light chain of a traditional two chain antibody have been joined to form one chain. Typically, a linker peptide is inserted between the two chains to allow for proper folding and creation of an active binding site.

The term “linker peptide” includes reference to a peptide within an antibody binding fragment (e.g., Fv fragment) which serves to indirectly bond the variable heavy chain to the variable light chain.

The term “contacting” includes reference to placement in direct physical association. With regard to this invention, the term refers to antibody-antigen binding.

As used herein, the term “anti-podoplanin” in reference to an antibody, includes reference to an antibody which is generated against podoplanin. In certain embodiments, the podoplanin is a primate podoplanin such as human podoplanin. In other embodiments, the antibody is generated against human podoplanin synthesized by a non-primate mammal after introduction into the animal of cDNA which encodes human podoplanin.

As used herein, “polypeptide”, “peptide” and “protein” are used interchangeably and include reference to a polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

The term “residue” or “amino acid residue” or “amino acid” includes reference to an amino acid that is incorporated into a protein, polypeptide, or peptide (collectively “peptide”). The amino acid can be a naturally occurring amino acid and, unless otherwise limited, can encompass known analogs of natural amino acids that can function in a similar manner as naturally occurring amino acids.

The amino acids and analogs referred to herein are described by shorthand designations as follows in Table 1:

TABLE 1
Amino Acid Nomenclature Name 3-letter 1-letter

| Alanine Ala A | Arginine Arg R | Asparagine Asn N |
|---|---|---|
| Glutamine Gln Q | Aspartic Acid Asp D | Cysteine Cys C |
| Homoserine Hse -- | Glutamic Acid Glu E | Histidine His H |
| Lysine Lys K | Glycine Gly G | Leucine Leu L |
| (0) | Isoleucine Ile I | Methionine sulfoxide |
| Methionine methylsulfonium Met (S—Me)— | | Met |
| Phenylalanine Phe F | Methionine Met M | Norleucine Nle - |
| Threonine Thr T | Proline Pro P | Serine Ser S |
| Valine Val V | Tryptophan Trp W | Tyrosine Tyr Y |

See also, Creighton, PROTEINS, W. H. Freeman and Company (1984).

A “conservative substitution”, when describing a protein refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups in Table 2 each contain amino acids that are conservative substitutions for one another:

TABLE 2

1) Alanine (A), Serine (S), Threonine (T);
2) Aspartic acid (D), Glutamic acid (E);
3) Asparagine (I), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
See also, Creighton, PROTEINS, W. H. Freeman and Company (1984).

The terms “substantially similar” in the context of a peptide indicates that a peptide comprises a sequence with at least 90%, preferably at least 95% sequence identity to the reference sequence over a comparison window of 10-20 amino acids. Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The phrase “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond. In the context of this invention, the cysteines which form the disulfide bond are within the framework regions of the single chain antibody and serve to stabilize the conformation of the antibody.

The terms “conjugating,” “joining,” “bonding” or “linking” refer to making two polypeptides into one contiguous polypeptide molecule. In the context of the present invention, the terms include reference to joining an antibody moiety to an effector molecule (EM). The linkage can be either by chemical or recombinant means. Chemical means refers to a reaction between the antibody moiety and the effector molecule such that there is a covalent bond formed between the two molecules to form one molecule.

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, under expressed or not expressed at all.

As used herein, “nucleic acid” or “nucleic acid sequence” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof as well as conservative variants, i.e., nucleic acids present in wobble positions of codons and variants that, when translated into a protein, result in a conservative substitution of an amino acid.

As used herein, “encoding” with respect to a specified nucleic acid, includes reference to nucleic acids which comprise the information for translation into the specified protein. The information is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Proc. Nat'l Acad. Sci. USA 82:2306-2309 (1985), or the ciliate Macronucleus, may be used when the nucleic acid is expressed in using the translational machinery of these organisms.

The phrase “fusing in frame” refers to joining two or more nucleic acid sequences which encode polypeptides so that the joined nucleic acid sequence translates into a single chain protein which comprises the original polypeptide chains.

As used herein, “expressed” includes reference to translation of a nucleic acid into a protein. Proteins may be expressed and remain intracellular, become a component of the cell surface membrane or be secreted into the extracellular matrix or medium.

By “host cell” is meant a cell which can support the replication or expression of the expression vector. Host cells may be prokaryotic cells such as E. coli, or eukaryotic cells such as yeast, insect, amphibian, or mammalian cells.

The phrase “phage display library” refers to a population of bacteriophage, each of which contains a foreign cDNA recombinantly fused in frame to a surface protein. The phage displays the foreign protein encoded by the cDNA on its surface. After replication in a bacterial host, typically E. coli, the phage which contain the foreign cDNA of interest are selected by the expression of the foreign protein on the phage surface.

“Sequence identity” in the context of two nucleic acid or polypeptide sequences includes reference to the nucleotides (or residues) in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers & Miller, Computer Applic. Biol. Sci. 4:11-17 (1988), e.g., as implemented in the program PC/GENE (Intelligenetics, Mountain View, Calif., USA). An indication that two peptide sequences are substantially similar is that one peptide is immunologically reactive with antibodies raised against the second peptide. Thus, a peptide is substantially similar to a second peptide, for example, where the two peptides differ only by a conservative substitution.

A “comparison window”, as used herein, includes reference to a segment of about 10-20 residues in which a sequence may be compared to a reference sequence of the same number of contiguous positions after the two sequences are optimally aligned. Methods of alignment of sequences for comparison are well known in the art. Optimal alignment of sequences for comparison may be conducted by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l Acad. Sci. USA 85:2444 (1988); by computerized implementations of these algorithms (including, but not limited to CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), Madison, Wis., USA); the CLUSTAL program is well described by Higgins & Sharp, Gene 73:237-244 (1988) and Higgins & Sharp, CABIOS 5:151-153 (1989); Corpet, et al., Nucl. Acids Res. 16:10881-90 (1988); Huang, et al., Computer Applications in the Biosciences 8:155-65 (1992); and Pearson, et al., Meth. in Molec. Biol. 24:307-31 (1994).

The terms “effective amount” or “amount effective to” or “therapeutically effective amount” include reference to a dosage of a therapeutic agent sufficient to produce a desired result, such as inhibiting cell protein synthesis by at least 50%, or killing the cell.

The term “therapeutic agent” includes any number of compounds currently known or later developed to act as anti-neoplastics, anti-inflammatories, cytokines, anti-infectives, enzyme activators or inhibitors, allosteric modifiers, antibiotics or other agents administered to induce a desired therapeutic effect in a patient.

The term “immunoconjugate” includes reference to a covalent linkage of an effector molecule to an antibody. The effector molecule can be an immunotoxin.

The term “toxin” includes reference to abrin, ricin, Pseudomonas exotoxin (PE), diphtheria toxin (DT), botulinum toxin, or modified toxins thereof. For example, PE and DT are highly toxic compounds that typically bring about death through liver toxicity. PE and DT, however, can be modified into a form for use as an immunotoxin by removing the native targeting component of the toxin (e.g., domain Ia of PE and the B chain of DT) and replacing it with a different targeting moiety, such as an antibody.

The term “in vivo” includes reference to inside the body of the organism from which the cell was obtained. “Ex vivo” and “in vitro” means outside the body of the organism from which the cell was obtained.

The phrase “malignant cell” or “malignancy” refers to tumors or tumor cells that are invasive and/or able to undergo metastasis, i.e., a cancerous cell.

As used herein, “mammalian cells” includes reference to cells derived from mammals including humans, rats, mice, guinea pigs, chimpanzees, or macaques. The cells may be cultured in vivo or in vitro.

### Anti-Podoplanin Antibodies

The present disclosure provides for antibodies targeted to podoplanin. Podoplanin, or PDPN, is a transmembrane sialoglycoprotein present on lymphatic endothelial cells. The immunoconjugates disclosed below target podoplanin using antibodies of the present disclosure. The antibodies are selectively reactive under immunological conditions to those determinant of podoplanin displayed on the surface of mammalian cells and are accessible to the antibody from the extracellular milieu.

The term “selectively reactive” includes reference to the preferential association of an antibody, in whole or part, with a cell or tissue bearing podoplanin and not to cells or tissues lacking podoplanin. It is, of course, recognized that a certain degree of non-specific interaction may occur between a molecule and a non-target cell or tissue. Nevertheless, selective reactivity, may be distinguished as mediated through specific recognition of podoplanin. Although selectively reactive antibodies bind antigen, they may do so with low affinity. On the other hand, specific binding results in a much stronger association between the antibody and cells bearing podoplanin than between the bound antibody and cells lacking podoplanin or low affinity antibody-antigen binding. Specific binding typically results in greater than 2-fold, preferably greater than 5-fold, more preferably greater than 10-fold and most preferably greater than 100-fold increase in amount of bound antibody (per unit time) to a cell or tissue bearing podoplanin as compared to a cell or tissue lacking podoplanin. Specific binding to a protein under such conditions requires an antibody that is selected for its specificity for a particular protein. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, ANTIBODIES, A LABORATORY MANUAL, Cold Spring Harbor Publications, New York (1988), for a description of immunoassay formats and conditions that can be used to determine specific immunoreactivity.

The term “immunologically reactive conditions” includes reference to conditions which allow an antibody generated to a particular epitope to bind to that epitope to a detectably greater degree than, and/or to the substantial exclusion of, binding to substantially all other epitopes Immunologically reactive conditions are dependent upon the format of the antibody binding reaction and typically are those utilized in immunoassay protocols or those conditions encountered in vivo. See Harlow & Lane, supra, for a description of immunoassay formats and conditions. Preferably, the immunologically reactive conditions employed in the methods of the present disclosure are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The anti-podoplanin antibodies employed in the present invention can be linked to effector molecules (EM) through the EM carboxyl terminus, the EM amino terminus, through an interior amino acid residue of the EM such as cysteine, or any combination thereof. Similarly, the EM can be linked directly to the heavy, light, Fc (constant region) or framework regions of the antibody. Linkage can occur through the antibody's amino or carboxyl termini, or through an interior amino acid residue. Further, multiple EM molecules (e.g., any one of from 2-10) can be linked to the anti-podoplanin antibody and/or multiple antibodies (e.g., any one of from 2-5) can be linked to an EM. The antibodies used in a multivalent immunoconjugate composition of the present invention can be directed to the same or different podoplanin epitopes.

In certain embodiments of the present disclosure, the anti-podoplanin antibody is a recombinant antibody such as a scFv or disulfide stabilized Fv antibody. Fv antibodies are typically about 25 kDa and contain a complete antigen-binding site with 3 CDRs per heavy and light chain. If the V_{H} and the V_{L} chain are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker.

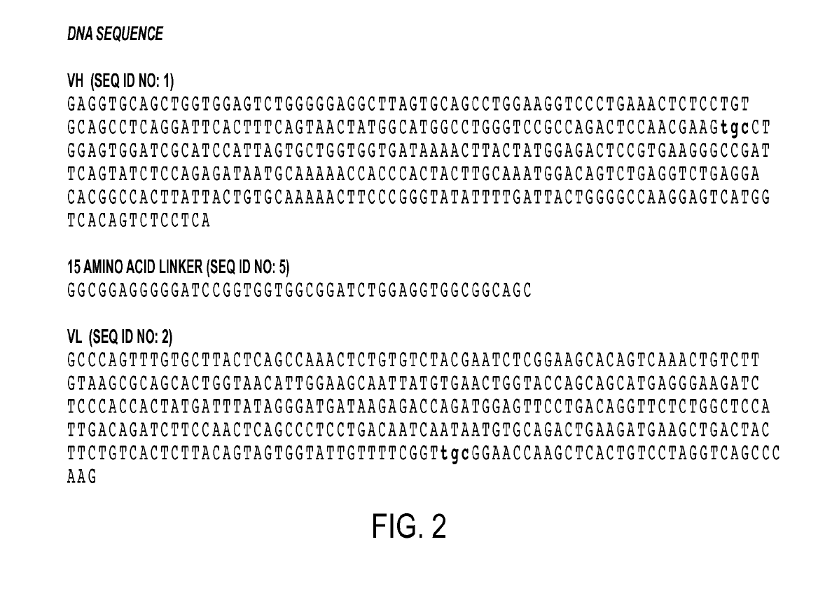

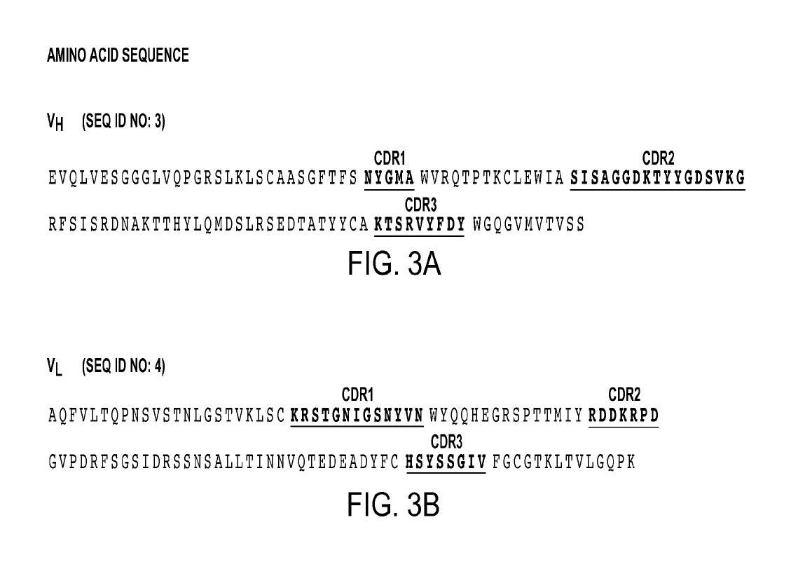

In other embodiments, the antibody is a single chain Fv (scFv). The V_{H} and the V_{L} regions of a scFv antibody comprise a single chain which is folded to create an antigen binding site similar to that found in two-chain antibodies. Once folded, noncovalent interactions stabilize the single chain antibody. In certain embodiments, the scFv is recombinantly produced. In yet another embodiment, the V_{H} region has the amino acid sequence as shown in FIG. 2. In another embodiment, the V_{H} region has the nucleic acid sequence as found in SEQ ID NO:1. In another embodiment, the V_{L} region has the amino acid sequence as found in FIG. 2. In another embodiment, the V_{L} region has the nucleic acid sequence as indicated in SEQ ID NO:2. In yet a further embodiment, the CDRs have the amino acid sequences as shown in FIG. 3. In another embodiment, the CDRs have the nucleic acid sequence as shown in SEQ ID NO: 3 and SEQ ID NO:4. One of skill will realize that conservative variants of the antibodies of the instant invention can be made. Such conservative variants employed in scFv fragments will retain critical amino acid residues necessary for correct folding and stabilizing between the V_{H} and the V_{L} regions. Conservatively modified variants of the prototype sequence of SEQ ID NO:1 have at least 80% sequence similarity, preferably at least 85% sequence similarity, more preferably at least 90% sequence similarity, and most preferably at least 95% sequence similarity at the amino acid level to its prototype sequence.

In some embodiments of the present invention, the scFv antibody is directly linked to the EM through the light chain. However, scFv antibodies can be linked to the EM via its amino or carboxyl terminus.

While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston, et al., Proc. Nat'l Acad. Sci. USA 8:5879 (1988); Bird, et al., Science 242:4236 (1988); Glockshuber, et al., Biochemistry 29:1362 (1990); U.S. Pat. No. 4,946,778, U.S. Pat. No. 5,132,405 and Stemmer, et al., Biotechniques 14:256-265 (1993), all incorporated herein by reference. Generally the peptide linker will have no specific biological activity other than to join the regions or to preserve some minimum distance or other spatial relationship between them. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is that shown as SEQ ID NO: 5 in FIG. 2. However, it is to be appreciated that some amino acid substitutions within the linker can be made. For example, a valine can be substituted for a glycine.

### Antibody Production

Methods of producing polyclonal antibodies are known to those of skill in the art. In brief, an immunogen, preferably isolated podoplanin or extracellular podoplanin epitopes are mixed with an adjuvant and animals are immunized with the mixture. When appropriately high titers of antibody to the immunogen are obtained, blood is collected from the animal and antisera are prepared. If desired, further fractionation of the antisera to enrich for antibodies reactive to the polypeptide is performed. See, e.g., Coligan, CURRENT PROTOCOLS IN IMMUNOLOGY, Wiley/Greene, N.Y. (1991); and Harlow & Lane, supra, which are incorporated herein by reference.

Monoclonal antibodies may be obtained by various techniques familiar to those skilled in the art. Description of techniques for preparing such monoclonal antibodies may be found in, e.g., Stites, et al. (eds.) BASIC AND CLINICAL IMMUNOLOGY (4TH ED.), Lange Medical Publications, Los Altos, Calif., and references cited therein; Harlow & Lane, supra; Goding, MONOCLONAL ANTIBODIES: PRINCIPLES AND PRACTICE (2D ED.), Academic Press, New York, N.Y. (1986); Kohler & Milstein, Nature 256:495-497 (1975); and particularly (Chowdhury, P. S., et al., Mol. Immunol. 34:9 (1997)), which discusses one method of generating monoclonal antibodies.

### Binding Affinity of Antibodies

The antibodies of this disclosure specifically bind to an extracellular epitope of podoplanin. An anti-podoplanin antibody has binding affinity for podoplanin if the antibody binds or is capable of binding podoplanin as measured or determined by standard antibody-antigen assays, for example, competitive assays, saturation assays, or standard immunoassays such as ELISA or RIA.

Such assays can be used to determine the dissociation constant of the antibody. The phrase “dissociation constant” refers to the affinity of an antibody for an antigen. Specificity of binding between an antibody and an antigen exists if the dissociation constant (k_{D}=1/K, where K is the affinity constant) of the antibody is <1 μM, preferably <100 nM, and most preferably <0.1 nM. Antibody molecules will typically have a kᵣ) in the lower ranges. k_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds. This method of defining binding specificity applies to single heavy and/or light chains, CDRs, fusion proteins or fragments of heavy and/or light chains, that are specific for podoplanin if they bind podoplanin alone or in combination.

### Immunoassays

The antibodies can be detected and/or quantified using any of a number of well recognized immunological binding assays (see, e.g., U.S. Pat. Nos. 4,366,241; 4,376,110; 4,517,288; and 4,837,168). For a review of the general immunoassays, see also METHODS IN CELL BIOLOGY, VOL. 37, Asai, ed. Academic Press, Inc. New York (1993); BASIC AND CLINICAL IMMUNOLOGY 7TH EDITION, Stites & Terr, eds. (1991). Immunological binding assays (or immunoassays) typically utilize a ligand (e.g., podoplanin) to specifically bind to and often immobilize an antibody. The antibodies employed in immunoassays of the present invention are discussed in greater detail supra.

Immunoassays also often utilize a labeling agent to specifically bind to and label the binding complex formed by the ligand and the antibody. The labeling agent may itself be one of the moieties comprising the antibody/analyte complex, i.e., the antipodoplanin antibody. Alternatively, the labeling agent may be a third moiety, such as another antibody, that specifically binds to the antibody/podoplanin protein complex.

In one aspect, a competitive assay is contemplated wherein the labeling agent is a second anti-podoplanin antibody bearing a label. The two antibodies then compete for binding to the immobilized podoplanin. Alternatively, in a non-competitive format, the podoplanin antibody lacks a label, but a second antibody specific to antibodies of the species from which the anti-podoplanin antibody is derived, e.g., murine, and which binds the antipodoplanin antibody, is labeled.

Other proteins capable of specifically binding immunoglobulin constant regions, such as Protein A or Protein G may also be used as the label agent. These proteins are normal constituents of the cell walls of streptococcal bacteria. They exhibit a strong non-immunogenic reactivity with immunoglobulin constant regions from a variety of species (see, generally Kronval, et al., J. Immunol. 111: 1401-1406 (1973); and Akerstrom, et al., J. Immunol. 135:2589-2542 (1985)).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antibody, volume of solution, concentrations, and the like. Usually, the assays will be carried out at ambient temperature, although they can be conducted over a range of temperatures, such as 10° C. to 40° C.

While the details of the immunoassays of the present invention may vary with the particular format employed, the method of detecting anti-podoplanin antibodies in a sample containing the antibodies generally comprises the steps of contacting the sample with an antibody which specifically reacts, under immunologically reactive conditions, to the podoplanin/antibody complex.

### Production of Immunoconjugates

Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents may include various drugs such as vinblastine, daunomycin and the like, cytotoxins such as native or modified Pseudomonas exotoxin or Diphtheria toxin, encapsulating agents, (e.g., liposomes) which themselves contain pharmacological compositions, radioactive agents such as ¹²⁵I, ³²P, ¹⁴C, ³H and ³⁵S and other labels, target moieties and ligands.

The choice of a particular therapeutic agent depends on the particular target molecule or cell and the biological effect is desired to evoke. Thus, for example, the therapeutic agent may be a cytotoxin which is used to bring about the death of a particular target cell. Conversely, where it is merely desired to invoke a non-lethal biological response, the therapeutic agent may be conjugated to a non-lethal pharmacological agent or a liposome containing a non-lethal pharmacological agent.

With the therapeutic agents and antibodies herein provided, one of skill can readily construct a variety of clones containing functionally equivalent nucleic acids, such as nucleic acids which differ in sequence but which encode the same EM or antibody sequence. Thus, the present invention provides nucleic acids encoding antibodies and conjugates and fusion proteins thereof.

### Recombinant Methods

The nucleic acid sequences of the present disclosure can be prepared by any suitable method including, for example, cloning of appropriate sequences or by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Lett. 22:1859-1862 (1981); the solid phase phosphoramidite triester method described by Beaucage & Caruthers, Tetra. Letts. 22(20):1859-1862 (1981), e.g., using an automated synthesizer as described in, for example, Needham-VanDevanter, et al. Nucl. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In another embodiment, the nucleic acid sequences of this disclosure are prepared by cloning techniques. Examples of appropriate cloning and sequencing techniques, and instructions sufficient to direct persons of skill through many cloning exercises are found in Sambrook, et al., MOLECULAR CLONING: A LABORATORY MANUAL (2ND ED.), Vols. 1-3, Cold Spring Harbor Laboratory (1989)), Berger and Kimmel (eds.), GUIDE TO MOLECULAR CLONING TECHNIQUES, Academic Press, Inc., San Diego Calif (1987)), or Ausubel, et al. (eds.), CURRENT PROTOCOLS IN MOLECULAR BIOLOGY, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Nucleic acids encoding native EM or anti-podoplanin antibodies can be modified to form the EM, antibodies, or immunoconjugates of the present invention. Modification by site-directed mutagenesis is well known in the art. Nucleic acids encoding EM or anti-podoplanin antibodies can be amplified by in vitro methods. Amplification methods include the polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well-known to persons of skill.

In another embodiment, immunoconjugates are prepared by inserting the cDNA which encodes an anti-podoplanin scFv antibody into a vector which comprises the cDNA encoding the EM. The insertion is made so that the scFv and the EM are read in frame, that is in one continuous polypeptide which contains a functional Fv region and a functional EM region. In one embodiment, cDNA encoding a diphtheria toxin fragment is ligated to a scFv so that the toxin is located at the carboxyl terminus of the scFv. In another embodiment, cDNA encoding PE is ligated to a scFv so that the toxin is located at the amino terminus of the scFv.

Once the nucleic acids encoding an EM, anti-podoplanin antibody, or an immunoconjugate of the present disclosure are isolated and cloned, one may express the desired protein in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eucaryotic cells such as the COS, CHO, HeLa and myeloma cell lines. No attempt to describe in detail the various methods known for the expression of proteins in prokaryotes or eukaryotes will be made. In brief, the expression of natural or synthetic nucleic acids encoding the isolated proteins of the invention will typically be achieved by operably linking the DNA or cDNA to a promoter (which is either constitutive or inducible), followed by incorporation into an expression cassette. The cassettes can be suitable for replication and integration in either prokaryotes or eukaryotes. Typical expression cassettes contain transcription and translation terminators, initiation sequences, and promoters useful for regulation of the expression of the DNA encoding the protein. To obtain high level expression of a cloned gene, it is desirable to construct expression cassettes which contain, at the minimum, a strong promoter to direct transcription, a ribosome binding site for translational initiation, and a transcription/translation terminator. For E. coli this includes a promoter such as the T7, trp, lac, or lambda promoters, a ribosome binding site and preferably a transcription termination signal. For eukaryotic cells, the control sequences can include a promoter and preferably an enhancer derived from immunoglobulin genes, SV40, cytomegalovirus, and a polyadenylation sequence, and may include splice donor and acceptor sequences. The cassettes of the invention can be transferred into the chosen host cell by well-known methods such as calcium chloride transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

One of skill would recognize that modifications can be made to a nucleic acid encoding a polypeptide of the present disclosure (i.e., anti-podoplanin antibody, PE, or an immunoconjugate formed from their combination) without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly H is) placed on either terminus to create conveniently located restriction sites or termination codons or purification sequences.

In addition to recombinant methods, the immunoconjugates, EM, and antibodies of the present disclosure can also be constructed in whole or in part using standard peptide synthesis. Solid phase synthesis of the polypeptides of the present invention of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, THE PEPTIDES: ANALYSIS, SYNTHESIS, BIOLOGY. VOL. 2: SPECIAL METHODS IN PEPTIDE SYNTHESIS, PART A. pp. 3-284; Merrifield, et al. J. Am. Chem. Soc. 85:2149-2156 (1963), and Stewart, et al., SOLID PHASE PEPTIDE SYNTHESIS, 2ND ED., Pierce Chem. Co., Rockford, Ill. (1984). Proteins of greater length may be synthesized by condensation of the amino and carboxyl termini of shorter fragments. Methods of forming peptide bonds by activation of a carboxyl terminal end (e.g., by the use of the coupling reagent N,N′-dicycylohexylcarbodiimide) are known to those of skill

### Purification

Once expressed, the recombinant immunoconjugates, antibodies, and/or effector molecules of the present invention can be purified according to standard procedures of the art, including ammonium sulfate precipitation, affinity columns, column chromatography, and the like (see, generally, R. Scopes, PROTEIN PURIFICATION, Springer—Verlag, N.Y. (1982)). Substantially pure compositions of at least about 90 to 95% homogeneity are preferred, and 98 to 99% or more homogeneity are most preferred for pharmaceutical uses. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well known and are applicable to the antibodies of this invention. See, Buchner, et al., Anal. Biochem. 205:263-270 (1992); Pluckthun, Biotechnology 9:545 (1991); Huse, et al., Science 246:1275 (1989) and Ward, et al., Nature 341:544 (1989), all incorporated by reference herein.

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena, et al., Biochemistry 9: 5015-5021 (1970), incorporated by reference herein, and especially described by Buchner, et al., supra.

Renaturation is typically accomplished by dilution (e.g, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. A preferred yield is obtained when these two proteins are mixed in a molar ratio such that a 5 fold molar excess of one protein over the other is not exceeded. It is desirable to add excess oxidized glutathione or other oxidizing low molecular weight compounds to the refolding solution after the redox-shuffling is completed.

### Pseudomonas Exotoxin and Other Toxins

Toxins can be employed with antibodies of the present disclosure to yield immunotoxins. Exemplary toxins include ricin, abrin, diphtheria toxin and subunits thereof, as well as botulinum toxins A through F. These toxins are readily available from commercial sources (e.g., Sigma Chemical Company, St. Louis, Mo.). Diptheria toxin is isolated from Corynebacterium diphtheriae. Ricin is the lectin RCA60 from Ricinus communis (Castor bean). The term also references toxic variants thereof. For example, see, U.S. Pat. Nos. 5,079,163 and 4,689,401. Ricinus communis agglutinin (RCA) occurs in two forms designated RCA.sub.60 and RCA.sub.120 according to their molecular weights of approximately 65 and 120 k_{D} respectively (Nicholson & Blaustein, J. Biochim. Biophys. Acta 266:543 (1972)). The A chain is responsible for inactivating protein synthesis and killing cells. The B chain binds ricin to cell-surface galactose residues and facilitates transport of the A chain into the cytosol (Olsnes, et al., Nature 249:627-631 (1974) and U.S. Pat. No. 3,060,165).

Abrin includes toxic lectins from Abrus precatorius. The toxic principles, abrin a, b, c, and d, have a molecular weight of from about 63 and 67 k_{D} and are composed of two disulfide-linked polypeptide chains A and B. The A chain inhibits protein synthesis; the B-chain (abrin-b) binds to D-galactose residues (see, Funatsu, et al., Agr. Biol. Chem. 52:1095 (1988); and Olsnes, Methods Enzymol. 50:330-335 (1978)).

In preferred embodiments of the present disclosure, the toxin is Pseudomonas exotoxin (PE). The term “Pseudomonas exotoxin” as used herein refers to a full-length native (naturally occurring) PE or a PE that has been modified. Such modifications may include, but are not limited to, elimination of domain Ia, various amino acid deletions in domains Ib, II and III, single amino acid substitutions and the addition of one or more sequences at the carboxyl terminus such as KDEL and REDL. See Siegall, et al., J. Biol. Chem. 264:14256 (1989). In a preferred embodiment, the cytotoxic fragment of PE retains at least 50%, preferably 75%, more preferably at least 90%, and most preferably 95% of the cytotoxicity of native PE. In a most preferred embodiment, the cytotoxic fragment is more toxic than native PE.

Native Pseudomonas exotoxin A (PE) is an extremely active monomeric protein (molecular weight 66 kD), secreted by Pseudomonas aeruginosa, which inhibits protein synthesis in eukaryotic cells. The native PE sequence is provided as SEQ ID NO:1 of U.S. Pat. No. 5,602,095, incorporated herein by reference. The method of action is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although a large part of it, amino acids 365-380, can be deleted without loss of cytotoxicity. See Siegall, et al., J. Biol. Chem. 264: 14256-14261 (1989), incorporated by reference herein.

PE employed in the present invention include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments of PE include PE40, PE38, and PE35. PE40 is a truncated derivative of PE as previously described in the art. See, Pai, et al., Proc. Nat'l Acad. Sci. USA 88:3358-62 (1991); and Kondo, et al., J. Biol. Chem. 263:9470-9475 (1988). PE35 is a 35 K_{D} carboxyl-terminal fragment of PE composed of a met at position 280 followed by amino acids 281-364 and 381-613 of native PE. In other embodiments, the cytotoxic fragment PE38 is employed. PE38 is a truncated PE pro-protein composed of amino acids 253-364 and 381-613 which is activated to its cytotoxic form upon processing within a cell (see U.S. Pat. No. 5,608,039, incorporated herein by reference).

In certain embodiment, PE38 is the toxic moiety of the immunotoxin of this disclosure, however, other cytotoxic fragments PE35 and PE40 are contemplated and are disclosed in U.S. Pat. Nos. 5,602,095 and 4,892,827, each of which is incorporated herein by reference.

### Conservatively Modified Variants of PE

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

The term “conservatively modified variants” applies to both amino acid and nucleic acid sequences. With respect to particular nucleic acid sequences, conservatively modified variants refer to those nucleic acid sequences which encode identical or essentially identical amino acid sequences, or if the nucleic acid does not encode an amino acid sequence, to essentially identical nucleic acid sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

### Other Therapeutic Moieties

Antibodies of the present disclosurecan also be used to target any number of different diagnostic or therapeutic compounds to cells expressing podoplanin on their surface. Thus, an antibody of the present invention, such as an anti-podoplanin scFv, may be attached directly or via a linker to a drug that is to be delivered directly to cells bearing podoplanin. Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an anti-podoplanin antibody may be an encapsulation system, such as a liposome or micelle that contains a therapeutic composition such as a drug, a nucleic acid (e.g. an antisense nucleic acid), or another therapeutic moiety that is preferably shielded from direct exposure to the circulatory system. Means of preparing liposomes attached to antibodies are well known to those of skill in the art. See, for example, U.S. Pat. No. 4,957,735; and Connor, et al., Pharm. Ther. 28:341-365 (1985).

### Detectable Labels

Antibodies of the present disclosure may optionally be covalently or noncovalently linked to a detectable label. Detectable labels suitable for such use include any composition detectable by spectroscopic, photochemical, biochemical, immunochemical, electrical, optical or chemical means. Useful labels in the present invention include magnetic beads (e.g. DYNABEADS), fluorescent dyes (e.g., fluorescein isothiocyanate, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g. polystyrene, polypropylene, latex, etc.) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Conjugation to the Antibody

In a non-recombinant embodiment of the invention, effector molecules, e.g., therapeutic, diagnostic, or detection moieties, are linked to the anti-podoplanin antibodies of the present disclosure using any number of means known to those of skill in the art. Both covalent and noncovalent attachment means may be used with anti-podoplanin antibodies of the present disclosure.

The procedure for attaching an effector molecule to an antibody will vary according to the chemical structure of the EM. Polypeptides typically contain variety of functional groups; e.g., carboxylic acid (COOH), free amine (—NH₂) or sulfhydryl (—SH) groups, which are available for reaction with a suitable functional group on an antibody to result in the binding of the effector molecule.

Alternatively, the antibody is derivatized to expose or attach additional reactive functional groups. The derivatization may involve attachment of any of a number of linker molecules such as those available from Pierce Chemical Company, Rockford Ill.

A “linker”, as used herein, is a molecule that is used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (e.g., through a disulfide linkage to cysteine). However, in certain embodiments, the linkers will be joined to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages which are cleavable in the vicinity of the target site. Cleavage of the linker to release the effector molecule from the antibody may be prompted by enzymatic activity or conditions to which the immunoconjugate is subjected either inside the target cell or in the vicinity of the target site. When the target site is a tumor, a linker which is cleavable under conditions present at the tumor site (e.g. when exposed to tumor-associated enzymes or acidic pH) may be used.

In view of the large number of methods that have been reported for attaching a variety of radiodiagnostic compounds, radiotherapeutic compounds, drugs, toxins, and other agents to antibodies one skilled in the art will be able to determine a suitable method for attaching a given agent to an antibody or other polypeptide.

### Pharmaceutical Compositions and Administration

The antibody and/or immunoconjugate compositions of this disclosure (i.e., PE linked to an anti-podoplanin antibody), are particularly useful for parenteral administration, such as intravenous administration or administration into a body cavity or lumen of an organ. For example, ovarian malignancies may be treated by intravenous administration or by localized delivery to the tissue surrounding the tumor.

The compositions for administration will commonly comprise a solution of the antibody and/or immunoconjugate dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

Thus, a typical pharmaceutical immunotoxin composition of the present invention for intravenous administration would be about 0.1 to 10 mg per patient per day. Dosages from 0.1 up to about 100 mg per patient per day may be used, particularly if the drug is administered to a secluded site and not into the circulatory or lymph system, such as into a body cavity or into a lumen of an organ. Actual methods for preparing administrable compositions will be known or apparent to those skilled in the art and are described in more detail in such publications as REMINGTON′S PHARMACEUTICAL SCIENCE, 19TH ED., Mack Publishing Company, Easton, Pa. (1995).

The compositions of the present invention can be administered for therapeutic treatments. In therapeutic applications, compositions are administered to a patient suffering from a disease, in an amount sufficient to cure or at least partially arrest the disease and its complications. An amount adequate to accomplish this is defined as a “therapeutically effective dose.” Amounts effective for this use will depend upon the severity of the disease and the general state of the patient's health. An effective amount of the compound is that which provides either subjective relief of a symptom(s) or an objectively identifiable improvement as noted by the clinician or other qualified observer.

Single or multiple administrations of the compositions are administered depending on the dosage and frequency as required and tolerated by the patient. In any event, the composition should provide a sufficient quantity of the proteins of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

Controlled release parenteral formulations of the immunoconjugate compositions of the present disclosure can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., COLLOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp. 315-339 (1992) both of which are incorporated herein by reference.

Polymers can be used for ion-controlled release of immunoconjugate compositions of the present invention. Various degradable and nondegradable polymeric matrices for use in controlled drug delivery are known in the art (Langer, R., Accounts Chem. Res. 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston, et al., Pharm. Res. 9:425-434 (1992); and Pec, et al., J. Parent. Sci. Tech. 44(2):58-65 (1990)). Alternatively, hydroxyapatite has been used as a microcarrier for controlled release of proteins (Ijntema, et al., Int. J. Pharm. 112:215-224 (1994)). In yet another aspect, liposomes are used for controlled release as well as drug targeting of the lipid-capsulated drug (Betageri, et at., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

Among various uses of the immunotoxins of the present invention are included a variety of disease conditions caused by specific human cells that may be eliminated by the toxic action of the fusion protein. One preferred application for the immunotoxins of the invention is the treatment of malignant cells expressing podoplanin. Exemplary malignant cells include astrocytomas, glioblastomas, melanoma and the like.

### Diagnostic Kits

In another embodiment, this invention provides for kits for the detection of podoplanin or an immunoreactive fragment thereof, (i.e., collectively, a “podoplanin protein”) in a biological sample. A “biological sample” as used herein is a sample of biological tissue or fluid that contains podoplanin. Such samples include, but are not limited to, tissue from biopsy, sputum, amniotic fluid, blood, and blood cells (e.g., white cells). Fluid samples may be of some interest, but are generally not preferred herein since detectable concentrations of podoplanin are rarely found in such a sample. Biological samples also include sections of tissues, such as frozen sections taken for histological purposes. A biological sample is typically obtained from a multicellular eukaryote, preferably a mammal such as rat, mice, cow, dog, guinea pig, or rabbit, and most preferably a primate such as macaques, chimpanzees, or humans.

Kits will typically comprise an anti-podoplanin antibody of the present disclosure. In some embodiments, the anti-podoplanin antibody will be an anti-podoplanin Fv fragment; preferably a scFv fragment.

In addition the kits will typically include instructional materials disclosing means of use of an antibody of the present disclosure (e.g. for detection of glioma cells in a sample). The kits may also include additional components to facilitate the particular application for which the kit is designed. Thus, for example, the kit may additionally contain means of detecting the label (e.g. enzyme substrates for enzymatic labels, filter sets to detect fluorescent labels, appropriate secondary labels such as a sheep anti-mouse-HRP, or the like). The kits may additionally include buffers and other reagents routinely used for the practice of a particular method. Such kits and appropriate contents are well known to those of skill in the art.

In one embodiment of the present disclosure, the diagnostic kit comprises an immunoassay. As described above, although the details of the immunoassays of the present disclosure may vary with the particular format employed, the method of detecting podoplanin in a biological sample generally comprises the steps of contacting the biological sample with an antibody which specifically reacts, under immunologically reactive conditions, to podoplanin. The antibody is allowed to bind to podoplanin under immunologically reactive conditions, and the presence of the bound antibody is detected directly or indirectly.

### EXAMPLES

Glioblastoma multiforme (GBM) is the most malignant and most frequently occurring brain tumor in the adult and pediatric populations. Podoplanin (PDPN) is a transmembrane sialoglycoprotein present on lymphatic endothelial cells. PDPN expression is upregulated in several cancers, including GBM. Cell surface expression analysis of 27 GBM xenografts demonstrated the glioma tumor antigen PDPN to be present at very high levels in 12 of the xenografts, which makes PDPN an ideal immunotherapeutic target for the treatment of GBMs. In the current study, we have constructed a recombinant single-chain antibody variable region fragment (scFv), NZ-1, specific for PDPN from the NZ-1 hybridoma. NZ-1 scFv was then fused to Pseudomonas exotoxin A, carrying a C-terminal KDEL peptide (NZ-1-PE38 KDEL). The immunotoxin (IT) was further stabilized by a disulfide bond between the heavy- and light-chain variable regions (dsNZ-1-PE38 KDEL). In vitro cytotoxicity of the NZ-1-PE38 KDEL and the dsNZ-1-PE38 KDEL ITs was measured in cells isolated from GBM xenografts, D534MG, D2159MG, D08-0499MG, D08-0493MG and D08-0466MG, and in the medulloblastoma cell line D581MED. The nonspecific ITs Anti-Tac-PE38 or P588-PE38 KDEL were used as controls. The dsNZ-1-PE38 KDEL IT was highly cytotoxic, with an IC50 in the range of 0.5-6.9 ng/mL on D534MG, D2159MG, D08-0499MG, D08-0493MG, D08-0466MG, and D581MED cells. The therapeutic potential of dsNZ-1-PE38-KDEL as a targeting agent for malignant gliomas will be assessed in vivo against PDPN expressing GBM xenograft models.

The cancer-targeting reagent, an immunotoxin comprising a Pseudomonas exotoxin (PE) attached to an Fv antibody fragment isolated from a podoplanin-specific monoclonal antibody IgG, targets only tumor cells expressing an unique human cancer marker but not normal cells. This reagent may be developed as a therapeutic drug for the treatment of malignant glioma, or malignant melanoma patients, or any malignant tumor expressing this cancer marker.

This cancer marker-specific immunotoxin can be used to treat malignant glioma patients, malignant melanoma patients, and patients with any type of cancer expressing this cancer marker based on the in vitro specific tumor cell-killing activity.

### Example 1

### Materials and Methods

1. Podoplanin and Antibody NZ-1:

Podoplanin/Aggrus is a mucin-like sialoglycoprotein that is highly expressed in malignant gliomas. Podoplanin has been reported to be a marker to enrich tumor-initiating cells, which are thought to resist conventional therapies and to be responsible for relapse. The purpose of this study is to determine whether an anti-podoplanin antibody is suitable to target malignant gliomas for subsequent therapy investigations. The binding affinity of an anti-podoplanin antibody, NZ-1 (rat IgG₂ₐ) was determined by surface plasmon resonance and Scatchard analysis. NZ-1 was radioiodinated with ¹²⁵I using Iodogen (¹²⁵I-NZ-1) or N-succinimidyl 4-guanidinomethyl 3-[¹³¹I]-iodobenzoate ([¹³¹I]SGMIB-NZ-1), and paired-label internalization assays of NZ-1 were performed. The tissue distribution of ¹²⁵I-NZ-1 and that of [¹³¹I]SGMIB-NZ-1 were then compared in athymic mice bearing glioblastoma xenografts. The dissociation constant (K_{D}) of NZ-1 was determined to be 1.2×10⁻¹⁰ M by surface plasmon resonance, and 9.8×10⁻¹⁰ M for D397MG glioblastoma cells by Scatchard analysis. Paired-label internalization assays in LN319 glioblastoma cells indicated that [¹³¹I]SGMIB-NZ-1 resulted in higher intracellular retention of radioactivity (26.3±0.79% of initially bound radioactivity at 8 hr) compared to that from the ¹²⁵I-NZ-1 (9.95±0.15% of initially bound radioactivity at 8 hr). Likewise, tumor uptake of [¹³¹I]SGMIB-NZ-1 (39.88±8.79% ID/g at 24 hr) in athymic mice bearing D2159MG pediatric GBM xenografts in vivo was significantly higher than that of ¹²⁵I-NZ-1 (29.72±6.05% ID/g at 24 hr). The overall results suggest that an anti-podoplanin antibody NZ-1 warrants further evaluation for antibody-based therapy against glioblastoma (Kato et al, NMB 2010).

2. Cell Surface Binding Determined by Flow Cytometry:

Indirect FACS analysis was performed with NZ-1 Mab vs control IgG2a. Briefly, 1×10⁶ cells were suspended in 500 ml of PBS containing 5% FBS (5% FBS/PBS). The NZ-1 or negative control was added to the cells at a concentration of 10 μg/ml and the samples were incubated for 40 min. After washing, cells were incubated with FITC-conjugated goat anti-rat IgG antibody (Zymed, South San Francisco, Calif.). To prevent internalization of target antigens during assays, all the reagents and buffers were kept on ice, and experiments were performed at 4° C. Stained cells were analyzed on a Becton Dickinson FACSort instrument equipped with CellQuest software (Becton Dickinson, San Jose, Calif.).

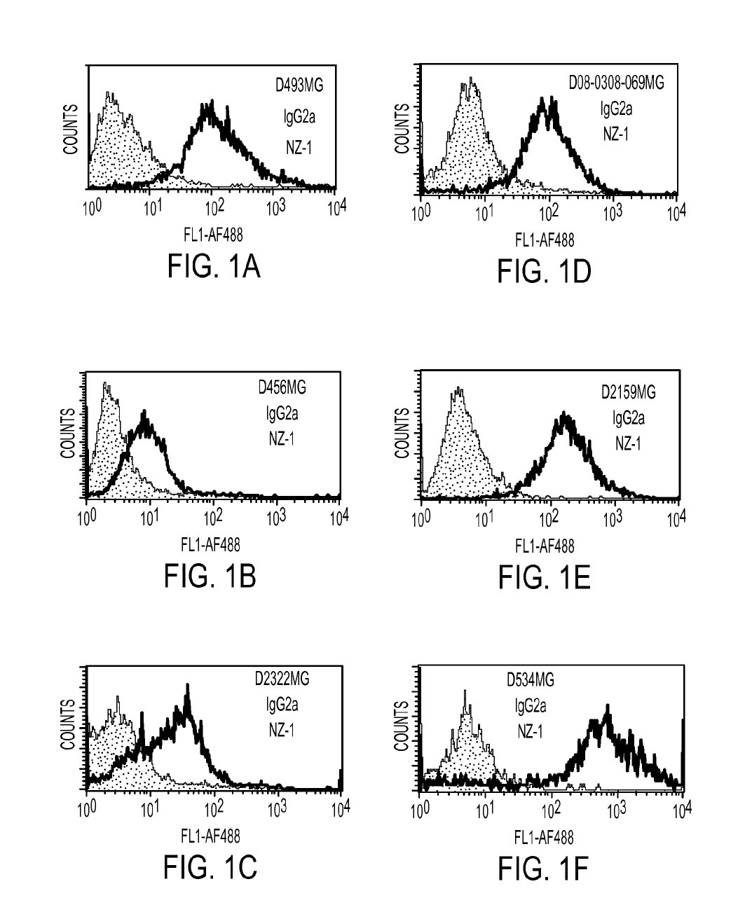

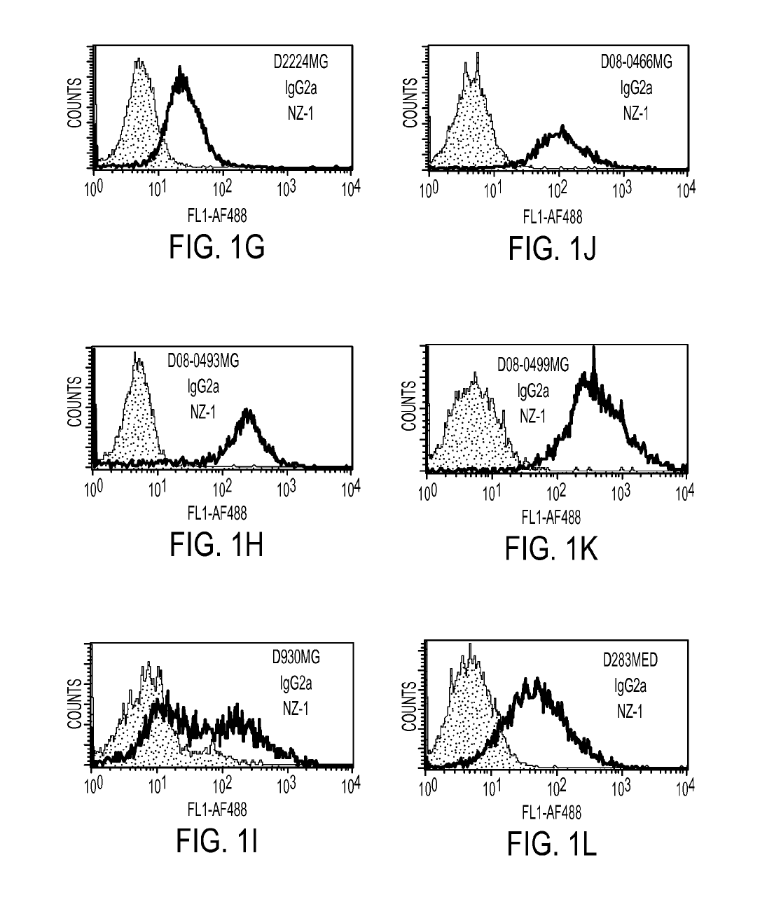

PDPN expression is upregulated in several cancers, including GBM. Cell surface expression analysis of 27 GBM xenografts demonstrated the glioma tumor antigen PDPN to be present at very high levels in 12 of the xenografts (FIG. 1), which makes PDPN an ideal immunotherapeutic target for the treatment of GBMs.

3. Cloning of Anti-PDPN scFv from NZ-1 mAb IgG Hybridoma:

Total cellular mRNA was isolated from 10⁶ hybridoma cells using Dynabeads, mRNA direct kit (Invitrogen, San Diego, Calif.). V_{H} and V_{L} cDNAs of the NZ-1 MAb was obtained by a RACE method using SMART RACE cDNA amplification kit (Clontech, Palo Alto, Calif.). In brief, adaptor-ligated cDNA was generated from 300 ng of the mRNA using PowerScript Reverse Transcriptase and SMART II A oligonucleotide (Clontech, Palo Alto, Calif.) along with 12 μM each of 3′ end primers designed to anneal the heavy chain (HC) and light chain (LC) constant region sequence of rat IgG2a immunoglobulin. The prepared cDNAs were used as the template for PCR reactions between 5′ end primer that binds to the adaptor sequence and the immunoglobulin HC and LC specific 3′ end primer specified above. The obtained sequences were aligned and verified according to the Kabat alignment scheme. The V_{H} domain was fused to the V_{L} domain by a 15 amino acid peptide (Gly₄Ser)₃ linker by PCR. The NZ-1 scFv fragment was cloned into pRK79 vector using T4 DNA ligase kit (Pierce Biotechnology, Rockford, Ill.). The NZ-1 (scdsFv) construct was obtained by mutating residues 44 of V_{H} and 103 of V_{L} by site directed mutagenesis using QuickChange Multi-Site Directed Mutagenesis Kit (Stratagene, La Jolla, Calif.). The DNA sequence and amino acid sequence of NZ-1 (scds)Fv were shown in FIG. 2 and FIG. 3, respectively.

4. Preparation of Recombinant Immunotoxins:

The NZ-1 scFv was used to generate immunotoxin by fusing with the sequences for domains II and III of Pseudomonas exotoxin A (PE38) or a variant carrying a C-terminal KDEL peptide for improved intracellular transport. The NZ-1-(scdsFv)-PE38 KDEL IT was obtained by ligating the NZ-1 (scdsFv) PCR fragment into pRB 199 vector and the sequence verified. The toxin was further purified as a monomer (64 kDa) by ion exchange and size exclusion chromatography to greater than 95% purity, and no dimer or aggregate was detected. Typically, the yields for the immunotoxin production were around 5%.

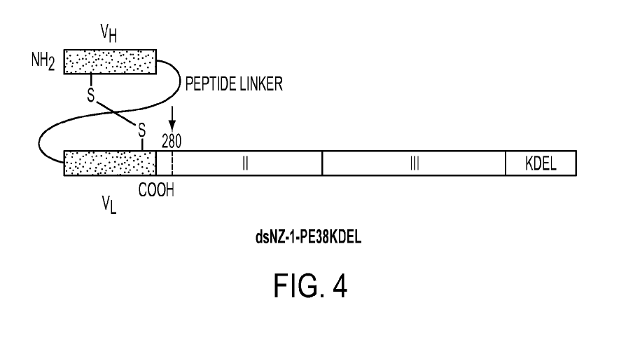

A recombinant single-chain antibody variable region fragment (scFv), NZ-1, specific for PDPN from the NZ-1 hybridoma has been constructed. NZ-1 scFv was then fused to Pseudomonas exotoxin A, carrying a C-terminal KDEL peptide (NZ-1-PE38 KDEL). The immunotoxin (IT) was further stabilized by a disulfide bond between the heavy- and light-chain variable regions (dsNZ-1-PE38 KDEL) and the schematic structure is shown in FIG. 4.

5. In Vitro Cell Killing Assay:

The cytotoxicity of the ITs on cultured cell lines and cells isolated from xenografts was assayed by inhibition of protein synthesis as described previously (Beers et al, 2000). Cells were seeded in 96-well plates at a density of 2×10⁴ cells per well in 200 μl of complete zinc option medium, 24 h before the assay. Immunotoxins were serially diluted to achieve a final concentration Of 0.01-1000 ng/ml in PBS containing 0.2% bovine serum albumin (BSA; 0.2% BSA/PBS), and 10 μl of diluted toxin was added to each well. Plates were incubated for 20 h at 37° C. and then pulsed with 1 μCi/well of L-[4,5-³H]leucine (Amersham Biosciences, Buckinghamshire, UK) in 25 μl of 0.2% BSA/PBS for 3 h at 37° C. Radiolabeled cells were captured on filter-mats and counted in a MicroBeta scintillation counter (PerkinElmer, Shelton, Conn.). The cytotoxic activity of an IT was defined by IC₅₀, which was the toxin concentration that suppressed incorporation of radioactivity by 50% as compared to the cells that were not treated with toxin.

### Example 2

### In Vitro Cytotoxicity

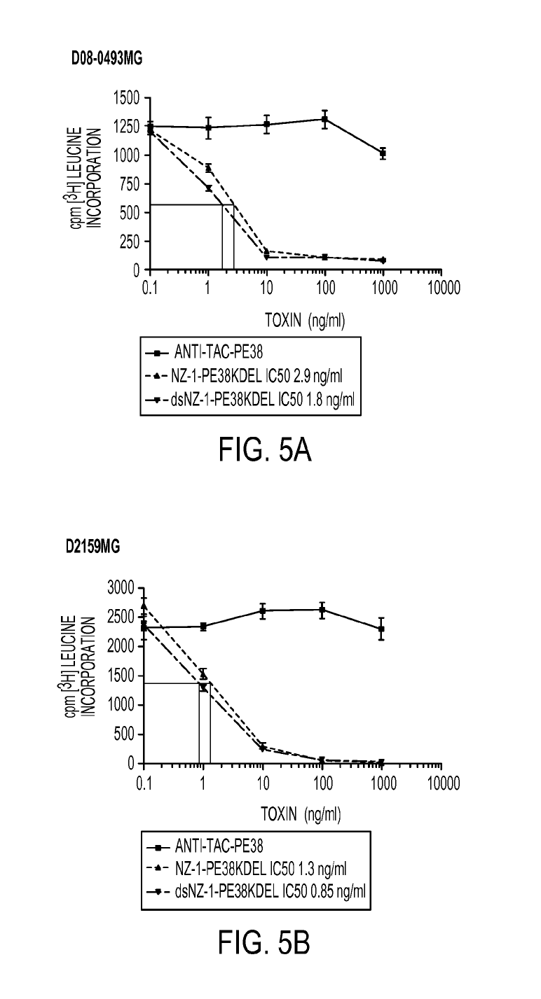

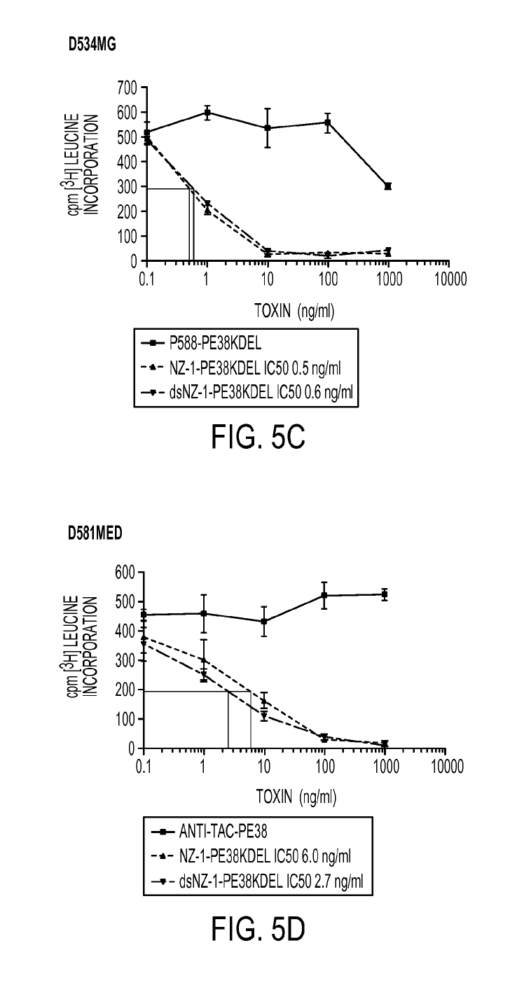

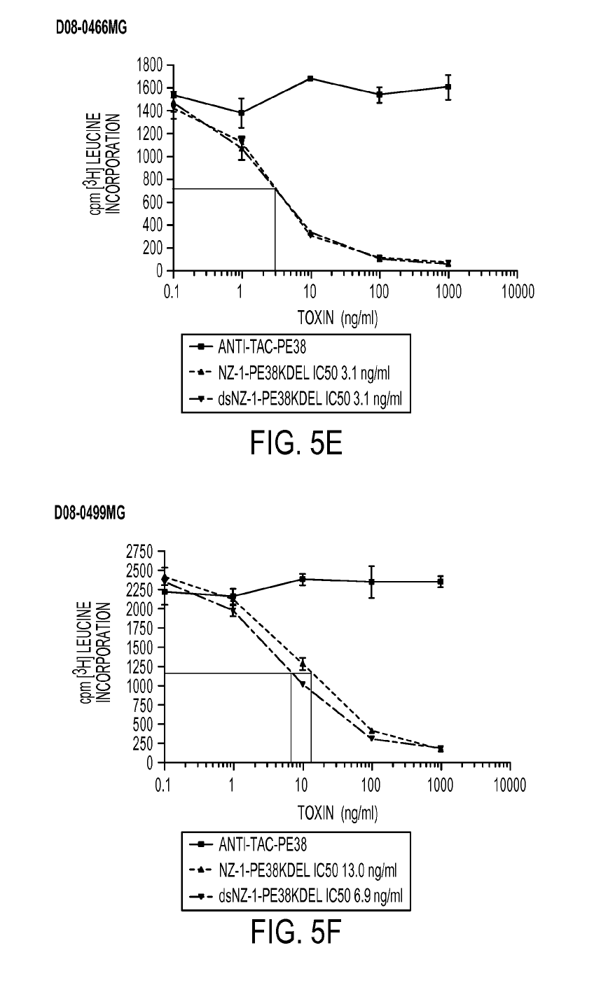

In vitro cytotoxicity of the NZ-1-PE38 KDEL and the dsNZ-1-PE38 KDEL ITs was measured in cells isolated from GBM xenografts, D534MG, D2159MG, D08-0499MG, D08-0493MG and D08-0466MG, and in the medulloblastoma cell line D581MED. The nonspecific ITs Anti-Tac-PE38 or P588-PE38 KDEL were used as controls. The dsNZ-1-PE38 KDEL IT was highly cytotoxic, with an IC50 in the range of 0.5-6.9 ng/mL on D534MG, D2159MG, D08-0499MG, D08-0493MG, D08-0466MG, and D581MED cells as shown in FIG. 5.

### Example 3

### Determination of Non-Specific Toxicity in Mice

The single dose mouse LD₄₀ will be determined by using female BALB/c mice (6-8 weeks old, 20 g), which will be given a single intra-peritoneal (i.p.) injection of different doses of NZ-1-(scdsFv)-PE38 KDEL IT (0.25-1.25 mg/kg) diluted in 200 μA of PBS containing 0.2% human serum albumin (PBS-HSA). Mice will be observed for 2 weeks following IT injection.

### Example 4

### In Vivo Tumor Model

Female athymic nude mice (approximately 20 g body weight, 4-6 week of age), can be injected sub-cutaneously (s.c.) with 3×10⁶ PDPN-positive cells suspended in 50 μl of PBS into the right flank. A total of 8-10 mice per arm can be randomly selected for inoculation when the implanted tumors reach a median tumor volume of 200-300 mm³ Mice can be treated with three doses of 0.3 mg/kg of NZ-1-(scdsFv)-PE38 KDEL IT or NZ-1-(scFv)-PE38 KDEL IT diluted in 0.2% PBS-HSA, by i.p. injections every other day. The control mice can be handled in the same manner and treated with 0.2% PBS-HAS or irrelevant immunotoxin, such as P588-PE38 KDEL. Tumors can be measured twice weekly with a handheld vernier caliper and the tumor volumes can be calculated in cubic millimeters by using the formula: ([length]×[width])/2. Animals can be tested out of the study when tumor volume meets both of the following criteria: 1) larger than 1000 mm³, and 2) 5 times its original treatment size.

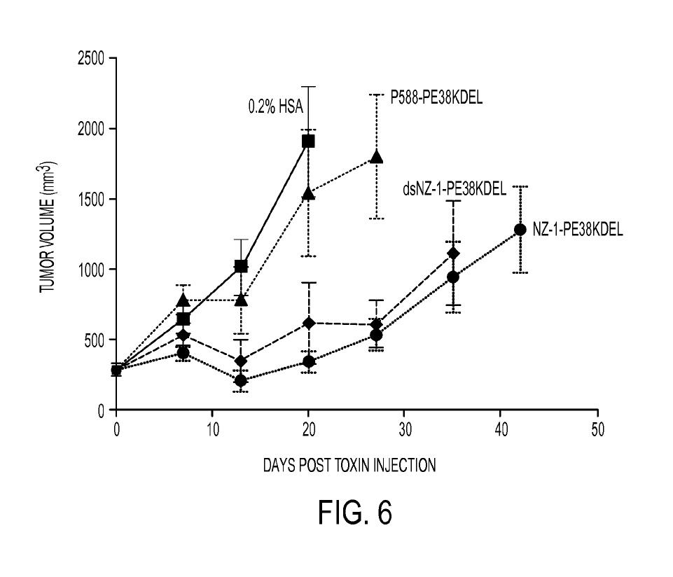

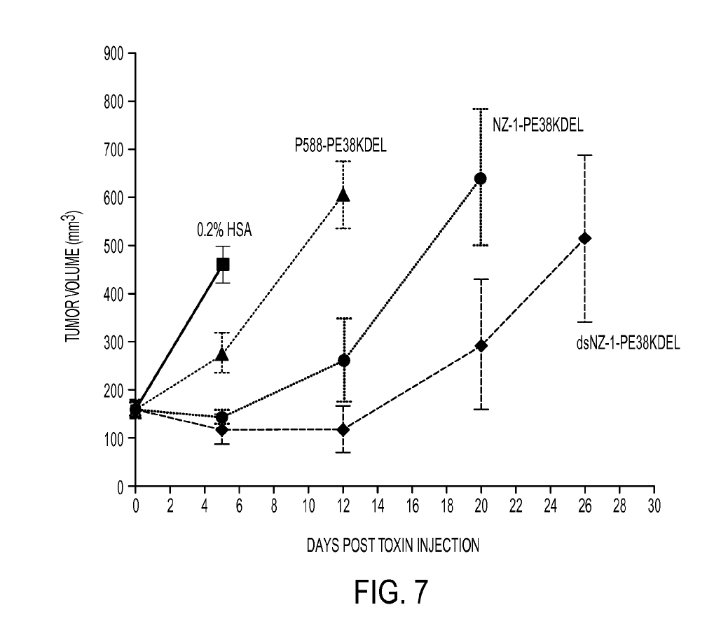

Data showing tumor volume growth inhibition in a medulloblastoma xengograft (D283MED) model and in a glioblastoma multiforme xenograft (D2159MG) model are shown in FIGS. 6 and 7, respectively. The positive effect of disulfide stabilization is also shown, particularly in the glioblastoma model.

Variations and modifications of the herein described systems, apparatuses, methods and other applications will undoubtedly suggest themselves to those skilled in the art. Accordingly, the foregoing description should be taken as illustrative and not in a limiting sense.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

We claim:

1. An immunotoxin which consists of a single polypeptide that binds to podoplanin and which is cytotoxic to cells expressing podoplanin, comprising:
an antibody heavy chain variable (“V_{H}”) region and an antibody light chain variable (“V_{L}”) region, each region comprising three complementarity determining regions (“CDRs”), wherein the CDRs of each region are numbered sequentially CDR1 to CDR3 starting from the amino terminus wherein CDR1, CDR2, and CDR3 of the V_{H} are shown in SEQ ID NO: 6, 7, and 8 respectively, and wherein CDR1, CDR2, and CDR3 of the V_{L} are as shown in SEQ ID NO: 9, 10 and 11; and
a Pseudomonas exotoxin or cytotoxic fragment thereof (“PE”).

2. The immunotoxin of claim 1 wherein the V_{H} chain of the immunotoxin is as shown in SEQ ID NO: 3.

3. The immunotoxin of claim 1 wherein the V_{L} chain of the immunotoxin is as shown in SEQ ID NO: 4.

4. The immunotoxin of claim 1 wherein the PE is PE38.

5. The immunotoxin of claim 1 wherein the PE is PE38 KDEL.

6. The immunotoxin of claim 1 which is disulfide stabilized.

7. The immunotoxin of claim 1 which is disulfide stabilized between a cysteine residue in the V_{H} at residue 44 of SEQ ID NO: 3 and a cysteine residue in the V_{L} at residue 103 of SEQ ID NO: 4.

8. The immunotoxin of claim 1 further comprising a linker segment between the V_{H} and the V_{L}.

9. The immunotoxin of claim 1 which is encoded by a molecule having a sequence in the 5′ to 3′ direction of SEQ ID NO: 1, 5, and 2.

10. A deoxyribonucleic acid molecule which encodes the immunotoxin of claim 1.

11. The deoxyribonucleic acid molecule of claim 10 wherein said molecule comprises a sequence in the 5′ to 3′ direction of SEQ ID NO: 1, SEQ ID NO: 5, and SEQ ID NO: 2.

12. A method of making an immunotoxin comprising, culturing a cell which comprises the deoxyribonucleic acid molecule of claim 10 in a cell medium, and collecting the immunotoxin from the cultured cells or cell medium.

13. A method of making an immunotoxin comprising, culturing a cell which comprises the deoxyribonucleic acid molecule of claim 11 in a cell medium, and collecting the immunotoxin from the cultured cells or cell medium.

14. A method for inhibiting the growth of malignant cells expressing podoplanin on their cell surface comprising:
contacting the malignant cells with an immunotoxin according to claim 1, whereby the immunotoxin inhibits the growth of the cells.

15. The method of claim 14 wherein the malignant cells are selected from the group consisting of astrocytoma cells, glioma cells, glioblastoma multiforme cells, melanoma cells and ependymoma cells.

16. The method of claim 14 wherein the contacting is performed in vitro.

17. The method of claim 14 wherein the contacting is performed in vivo.

18. The method of claim 14 wherein the V_{H} chain of the immunotoxin is as shown in SEQ ID NO: 3.

19. The method of claim 14 wherein the V_{L} chain of the immunotoxin is as shown in SEQ ID NO: 4.

20. The method of claim 14 wherein the PE is PE38.

21. The method of claim 14 wherein the PE is PE38 KDEL.

22. The method of claim 14 wherein the immunotoxin is disulfide stabilized.

23. The method of claim 14 wherein the immunotoxin is disulfide stabilized between a cysteine residue in the V_{H} at residue 44 of SEQ ID NO: 3 and in the V_{L} at residue 103 of SEQ ID NO: 4.

24. The method of claim 14 wherein the immunotoxin further comprises a linker segment between the V_{H} and the V_{L}.

25. The method of claim 14 wherein the immunotoxin is encoded by a molecule having a sequence in the 5′ to 3′ direction of SEQ ID NO: 1, 5, and 2.
